(19) United States
(12) Patent Application Publication
(10) Pub. No.: US 2022/0401266 A1
(43) Pub. Date: Dec. 22, 2022

(54) MULTI-LAYER WOUND DRESSING ASSEMBLIES

(71) Applicant: Nevad Medical Solutions LLC., Reno, NV (US)

(72) Inventors: Billie Joyce Adams, Reno, NV (US); Cooper Smith, Reno, NV (US)

(21) Appl. No.: 17/806,568

(22) Filed: Jun. 13, 2022

Related U.S. Application Data
(60) Provisional application No. 63/202,724, filed on Jun. 22, 2021.

(51) Int. Cl. A61F 13/00; A61F 13/02

(52) U.S. Cl. CPC A61F 13/00068; A61F 13/0203; A61M 2205/3324

(57) ABSTRACT

A multi-layer wound dressing comprises a carrier film comprising a transparent material or a semi-transparent material, and an adhesive layer comprising a silicone-based material and in contact with the carrier film and exhibiting a tackiness formulated and configured to decrease over a duration of time. Related multi-layer wound dressings are also disclosed.

### CROSS-REFERENCE TO RELATED APPLICATION

This application claims the benefit under 35 U.S.C. § 119(e) of U.S. Provisional Patent Application Ser. No. 63/202,724, filed Jun. 22, 2021, the disclosure of which is hereby incorporated herein in its entirety by this reference.

### FIELD

Embodiments of the disclosure relate generally to multi-layer wound dressings. More particularly, embodiments of the disclosure relate to multi-layer wound dressings including adhesive materials formulated and configured to exhibit a reduction in tackiness over a duration of time.

### BACKGROUND

A dressing is a pad or compress applied to a wound to promote healing and protect the wound from further harm. Dressings have a number of different purposes including, for example, providing a physical barrier between the wound and an external environment, stemming bleeding (e.g., sealing the wound to expedite the blood clotting process), protecting the wound from infection, absorbing exudate (e.g., soaking blood, plasma, and other fluids), and easing pain. Dressings are applied in direct contact with the wound, as opposed to bandages, which are most often used to hold a dressing in place. As there are many functions of dressings, there are many types of wound dressings formed from different materials. For example, wound dressings include gauze sponges, gauze bandage rolls, non-adherent pads, non-adherent wet dressings, foam dressings, and calcium alginates. Many wound dressings are self-adhesive. Some wound dressings include a pressure sensitive adhesive. However, application of pressure to adhere the wound dressing to the patient may unnecessarily cause discomfort to the patient.

The type of wound dressing applied to a wound depends on the type of wound to which the dressing is applied. Most invasive surgical procedures require a dressing to cover the incision site. The purpose of the dressing is to protect the incision site by creating a physical barrier, keeping the incision site moist, and preventing a surgical site infection (SSI). The dressing is secured in place to keep the dressing from moving and protect the wound. Normally, the dressing is held in place with a high-tack adhesive. Current wound dressings cover and protect wounds, but are difficult to remove and can cause further injury or discomfort during removal.

### BRIEF SUMMARY

In accordance with one embodiment described herein, a multi-layer wound dressing assembly comprises a carrier film comprising a transparent material or a semi-transparent material, and an adhesive layer comprising a silicone-based material and in contact with the carrier film and exhibiting a tackiness formulated and configured to decrease over a duration of time.

In additional embodiments, a multi-layer wound dressing comprises a carrier film comprising a transparent or semi-transparent material, and an adhesive layer in contact with the carrier film and exhibiting a tackiness formulated and configured to decrease over a duration of time.

In yet additional embodiments, a multi-layer wound dressing assembly comprises a transparent carrier film, a transparent adhesive layer on a side of the transparent carrier film, and one or more absorbent materials on a side of the transparent adhesive layer opposite the transparent carrier film.

In further embodiments, a multi-layer wound dressing assembly comprises one or more first absorbent materials, a first substantially transparent adhesive layer overlying the one or more first absorbent materials, a first substantially transparent carrier film overlying the first substantially transparent adhesive layer, one or more second absorbent materials overlying the first substantially transparent carrier firm, a second substantially transparent adhesive layer overlying the one or more second absorbent materials, and a second substantially transparent carrier film overlying the second substantially transparent adhesive layer.

### BRIEF DESCRIPTION OF THE SEVERAL VIEWS OF THE DRAWINGS

FIG. 1 is a simplified partial cross-sectional view of a multi-layer wound dressing assembly, in accordance with embodiments of the disclosure;

FIG. 2 is a simplified partial cross-sectional view of a multi-layer wound dressing assembly, in accordance with additional embodiments of the disclosure;

FIG. 3A is a simplified partial cross-sectional view of a multi-layer wound dressing assembly, in accordance with embodiments of the disclosure;

FIG. 3B is a simplified partial cross-sectional view of a portion of the multi-layer wound dressing assembly of FIG. 3A, in accordance with embodiments of the disclosure;

FIG. 3C is a simplified partial top-down view of the multi-layer wound dressing assembly of FIG. 3A, in accordance with embodiments of the disclosure;

FIG. 3D is a simplified partial top-down view of the multi-layer wound dressing assembly of FIG. 3A, in accordance with embodiments of the disclosure;

FIG. 3E and FIG. 3F are simplified partial cross-sectional views illustrating different embodiments of the protrusions of an adhesive layer of the multi-layer wound dressing assembly of FIG. 3A, in accordance with additional embodiments of the disclosure;

FIG. 4A and FIG. 4B are a respective simplified partial cross-sectional view and a simplified partial top-down view of a multi-layer wound dressing assembly, in accordance with other embodiments of the disclosure; and

FIG. 5 is a simplified partial cross-sectional view of a multi-layer wound dressing assembly, in accordance with further embodiments of the disclosure.

### DETAILED DESCRIPTION

The following description provides specific details, such as material types and dimensions order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional fabrication techniques employed in the industry. Also note, any drawings accompanying the present application are for illustrative purposes only, and are thus not drawn to scale. Additionally, elements common between figures may retain the same numerical designation.

According to embodiments described herein, a multi-layer wound dressing comprises one or more adhesive layers formulated and configured to exhibit a reduction in tackiness (e.g., adhesiveness) over a duration of time to facilitate maintenance of the multi-layer wound dressing over a wound and removal of the multi-layer wound dressing from a patient without causing tearing or bruising, or re-opening the wound or incision. One or more components of the multi-layer wound dressing may comprise a transparent material or a semi-transparent material to facilitate visual inspection of the wound without removal of the multi-layer wound dressing. In some embodiments, the one or more adhesive layers are configured to exhibit a reduction in tackiness responsive to exposure to one or more conditions, such as electromagnetic radiation, water, a solvent, or a change in temperature (e.g., heating or cooling). The reduction in tackiness of the multi-layer wound dressing facilitates removal of the multi-layer wound dressing from the patient without causing discomfort or further injury. In some embodiments, the multi-layer wound dressing is configured to cover a wound without sutures. In some embodiments, the multi-layer wound dressing includes a transparent or semi-transparent adhesive layer. One or more absorbent materials configured to contact the wound contact at least some surfaces of the adhesive layer and at least other surfaces of the adhesive layer are not in contact with the one or more absorbent materials such that the wound may be viewed by an observer (e.g., a health care provider, the patient) through the adhesive material. In use and operation, the wound may not be covered by the one or more absorbent materials and the wound may not be directly contacted by the one or more absorbent materials. In some embodiments, a portion of the adhesive layer configured to be in contact with the patient includes a textured surface defining channels through which liquid from the wound may travel and be absorbed by the one or more absorbent materials.

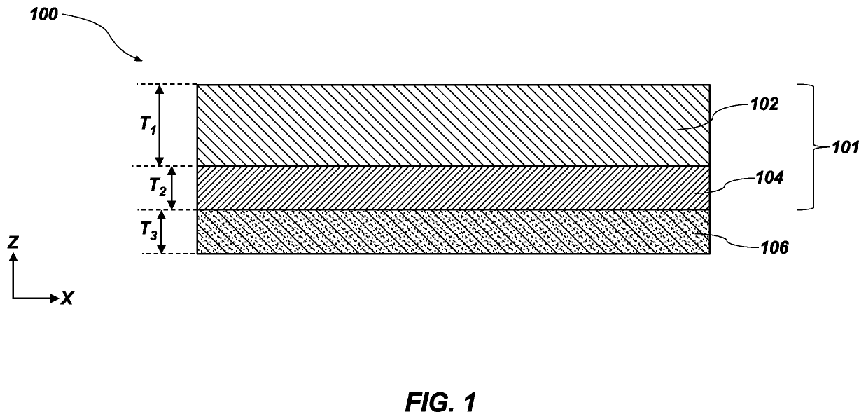

FIG. 1 is a simplified partial cross-sectional view of a multi-layer wound dressing assembly 100, in accordance with embodiments of the disclosure. The multi-layer wound dressing assembly 100 may be applied to a wound, an incision, or another portion of an individual (e.g., a patient) to be protected. In some embodiments, the multi-layer wound dressing assembly 100 is configured for use post-operation and may remain on a patient while in the hospital and/or after leaving the hospital.

The multi-layer wound dressing assembly 100 comprises a multi-layer wound dressing 101 comprising a carrier film 102 and an adhesive layer 104 vertically underlying (e.g., in the Z-direction) and in contact with the carrier film 102. The multi-layer wound dressing assembly 100 further comprises a sacrificial liner 106 vertically underlying the adhesive layer 104. In some embodiments, the adhesive layer 104 is between (e.g., vertically between) the carrier film 102 and the sacrificial liner 106.

The carrier film 102 may comprise a flexible and inelastic material configured to hold the multi-layer wound dressing 101 over a wound of the patient while covering and protecting the wound (e.g., incision site) from the external environment. The flexibility of the carrier film 102 facilitates conforming the multi-layer wound dressing 101 to the body of the patient around the wound. By way of non-limiting example, in some embodiments, the carrier film 102 has a thickness such that it may be conformed to a body of a patient. In some such embodiments, the carrier film 102 may not be elastic (e.g., is inelastic). In other words, under tension or strain, the carrier film 102 may exhibit a relatively high yield point and may exhibit a permanent deformation at the relatively high strain. In other embodiments, the carrier film 102 is elastic.

The carrier film 102 may comprise a transparent or a semi-transparent material. In some embodiments, the carrier film 102 comprises a transparent material. In some such embodiments, the carrier film 102 may be formulated and configured to facilitate substantial transmission (e.g., substantially complete transmission) of electromagnetic radiation (e.g., within the visible spectrum) therethrough such that underlying materials (e.g., the adhesive layer 104, the wound) are visible through the carrier film 102. In some embodiments, the carrier film 102 comprises a semi-transparent material and the underlying materials are partially visible through the carrier film 102. In some embodiments, the carrier film 102 comprises polyethylene terephthalate (PET) and is transparent or semi-transparent. In some embodiments, an amount of transparency of the carrier film 102 may be based on a surface roughness of the carrier film 102 (e.g., whether the surface of the carrier film 102 includes wrinkles, bends, folding, etc.). In other embodiments, the carrier film 102 comprises an opaque (e.g., non-transparent) material.

Because the carrier film 102 is transparent or semi-transparent, in use and operation, the wound of the patient may be visibly checked for infections and healing without removal of the multi-layer wound dressing 101. Accordingly, checking the wound may be accomplished without damaging or hindering the healing process and without unnecessarily exposing a potentially open wound to the environment and further risk of infection. In other words, the wound may be checked without removing the multi-layer wound dressing 101 from the patient since the wound may be visibly checked through the carrier film 102 and the adhesive layer 104.

In some embodiments, the carrier film 102 comprises a substantially impermeable (e.g., non-breathable, non-porous) material such that the carrier film 102 is substantially impermeable to the external environment. In some embodiments, the carrier film 102 is substantially impermeable to moisture (e.g., water), air, or other environmental conditions. In some such embodiments, the carrier film 102 substantially protects the wound from external elements that may cause infection. In addition, moisture between the carrier film 102 and the wound is sealed by the carrier film 102, maintaining the moisture of the wound.

A thickness T₁ of the carrier film 102 in the vertical direction (e.g., the Z-direction) may be within a range of from about 0.01 millimeter (mm) to about 10.0 mm, such as from about 0.01 mm to about 0.1 mm, from about 0.1 mm to about 0.5 mm, from about 0.5 mm to about 1.0 mm, from about 1.0 mm to about 2.0 mm, from about 2.0 mm to about 3.0 mm, from about 3.0 mm to about 5.0 mm, or from about 5.0 mm to about 10.0 mm. In some embodiments, the thickness T₁ of the carrier film 102 is within a range of from about 50 μm to about 100 μm.

The adhesive layer 104 may directly contact the carrier film 102. The adhesive layer 104 may comprise a material exhibiting a desired tackiness (e.g., adhesiveness) to secure the multi-layer wound dressing 101 to the patient. In some embodiments, the adhesive layer 104 comprises an adhesive gel. In some embodiments, the carrier film 102 is adhered to the adhesive layer 104.

The adhesive layer 104 may be formulated and configured to exhibit a relatively high tackiness when initially applied to a patient. The tackiness of the adhesive layer 104 may decrease over a duration of time to facilitate removal of the multi-layer wound dressing 101 after the desired duration of time has passed. In some embodiments, the adhesive layer 104 is formulated and configured to exhibit a predetermined reduction in tackiness over a predetermined duration of time, depending on the use of the multi-layer wound dressing 101. In other embodiments, the adhesive layer 104 comprises a material composition configured to exhibit a decrease in tackiness responsive to exposure to one or more conditions, such as electromagnetic radiation, water, a solvent, or a change in temperature (e.g., heating or cooling (e.g., freezing)). In some such embodiments, the adhesive layer 104 may exhibit a reduction in tackiness over a duration or may exhibit a substantially uniform tackiness until exposed to the one or more conditions.

Reducing the tackiness of the adhesive layer 104 over a duration of time and/or responsive to exposure to one or more conditions facilitates removal of the adhesive layer 104 and the multi-layer wound dressing 101 from the patient and reduces the pain, discomfort, and risk of bruising and re-opening the wound or incision during removal of the multi-layer wound dressing 101 from the patient. Accordingly, the adhesive layer 104 exhibits a high tackiness when initially applied to the patient to hold, close, and protect the wound and is formulated and configured to transition from the high tackiness to a relatively lower tackiness over the duration of time, responsive to exposure to the one or more conditions, or both to facilitate removal of the adhesive layer 104 and the associated multi-layer wound dressing 101. The rate at which the adhesive layer 104 loses tackiness is selected based on the application or amount of time the multi-layer wound dressing 101 is intended to be adhered to the skin of the patient.

In some embodiments, the tackiness of the adhesive layer 104 when initially applied to a patient is greater than about 55 grams (e.g., greater than about 100 grams) defined using ANSI D1876 peel resistance test. In some embodiments, the tackiness of the adhesive layer 104 decreases to less than about 50 grams using the ANSI D1876 peel resistance test when the adhesive layer 104 is to be removed. In some embodiments, the tackiness of the adhesive layer 104 of the multi-layer wound dressing assembly 100 prior to removal of the sacrificial liner 106 and placement of the multi-layer would dressing 101 on a patient is about 55 grams using the ANSI D1876 peel resistance test.

In some embodiments, the duration of time over which the adhesive layer 104 decreases in tackiness is within a range of from about 6 hours to about 14 days, such as from about 6 hours to about 12 hours, from about 12 hours to about 24 hours, from about 1 day to about 2 days, from about 2 days to about 3 days, from about 3 days to about 5 days, from about 5 days to about 7 days, from about 7 days to about 10 days, or from about 10 days to about 14 days. In some embodiments, a half-life of the tackiness is within a range of from about 6 hours to about 14 days, such as from about 6 hours to about 12 hours, from about 12 hours to about 24 hours, from about 1 day to about 2 days, from about 2 days to about 3 days, from about 3 days to about 5 days, from about 5 days to about 7 days, from about 7 days to about 10 days, or from about 10 days to about 14 days. However, the disclosure is not so limited and the duration and half-life may be different than those described above.

In some embodiments, such as for short-term applications (e.g., post-surgical incision protection), the duration is less than about 24 hours. In other embodiments, the duration is greater than 1 day and less than 3 days, such as from about 2 days to about 3 days. In other embodiments, the duration is greater than 3 days, such as greater than 5 days, or greater than 7 days, such as for use after a hospital stay. In some embodiments, the adhesive layer 104 reduces in tackiness at a substantially predetermined (e.g., predictable) rate over the duration until it reaches a tackiness such that the adhesive layer 104 is removed from the patient while reducing (e.g., minimizing) pulling or damaging the patient's skin. In some embodiments, the adhesive layer 104 does not substantially reduce in tackiness until a predetermined time or until the adhesive layer 104 is exposed to one or more conditions (e.g., a solvent, water, ultraviolet radiation, a predetermined temperature (e.g., heating or cooling, such as freezing)). In some embodiments, a tackiness of the adhesive layer 104 is reduced by exposure to (e.g., absorption of) moisture, exposure to UVA and/or UVB light, or natural decay of at least one component of the adhesive layer 104 through a continued chemical reaction based on the composition of the adhesive layer 104. In some embodiments, the tackiness of the adhesive layer 104 is reduced by exposure to a solvent, such as ethyl chloride. In some embodiments, as described in further detail herein, one or more components of the multi-layer wound dressing 101 (e.g., the adhesive layer 104) includes one or more channels to facilitate contact of the adhesive layer 104 with a solvent to reduce the tackiness of the adhesive layer 104.

In some embodiments, the adhesive layer 104 is substantially transparent. In other embodiments, the adhesive layer 104 is substantially semi-transparent. In some embodiments, the adhesive layer 104 is formulated and configured to facilitate substantial transmission (e.g., substantially complete transmission) of electromagnetic radiation (e.g., within the visible spectrum) therethrough such that underlying materials (e.g., the wound) are visible through the adhesive layer 104.

In some embodiments, the adhesive layer 104 is formulated and configured to exhibit a reduction in tackiness responsive to exposure to one or more conditions, such as exposure to one or more of electromagnetic radiation (e.g., electromagnetic radiation within a particular range of wavelengths, such as ultraviolet (UV) radiation), exposure to moisture (e.g., water), exposure to one or more solvents, heating (e.g., a temperature greater than about 40° C.), and cooling (e.g., a temperature less than about 30° C., such as less than about 25° C.). In some embodiments, the adhesive layer 104 is configured to exhibit a reduction in tackiness when frozen (e.g., such as by exposure to dimethyl ether, tetrafluorethane, or another material). In some embodiments, the adhesive layer 104 comprises a water-soluble material. In some such embodiments, the tackiness of the adhesive layer 104 may be reduced by exposure to water.

The adhesive layer 104 may comprise an adhesive gel. In some embodiments, the adhesive layer 104 is formed of and includes a material comprising carbon atoms, oxygen atoms, and silicon atoms. In some embodiments, the adhesive layer 104 comprises a silicone-based (e.g., a polysiloxane) material. In some embodiments, the adhesive layer 104 comprises a liquid silicon material (e.g., a liquid silicon rubber). In some such embodiments, the silicone-based material comprises the chemical formula, —R₂Si—O—SiR₂—, wherein R is an organic group and may include one or more functional groups. In some embodiments, each of the R groups comprises substantially the same material (e.g., functional group). In other embodiments, at least one of the R groups is different than at least one of the other R groups. The R groups bonded to the same silicon atom may comprise the same group or may comprise different groups.

The R groups may individually comprise one or more of methyl groups, vinyl groups, phenyl groups, trifluoropropyl groups, and fluoro groups (e.g., fluoroalkyl groups).

In some embodiments, the R groups individually comprise one or more methyl groups and the silicone-based material of the adhesive layer 104 comprises a dimethylsilicone elastomer (also referred to as “methyl silicone rubber”).

In some embodiments, the R groups individually comprise one or more methyl groups and one or more phenyl groups and the silicone-based material of the adhesive layer 104 comprises a methyl-phenylsilicone elastomer (also referred to as “phenylsilicone rubber”).

In some embodiments, the R groups individually comprise one or more methyl groups and one or more vinyl groups and the silicone-based material of the adhesive layer 104 comprises a methylvinylsilicone elastomer.

In some embodiments, the R groups individually comprise one or more methyl groups and one or more phenyl groups and the silicone-based material of the adhesive layer 104 comprises a methyl-phenylsilicone elastomer (also referred to as “phenylsilicone rubber”).

In some embodiments, the R groups individually comprise one or more methyl groups, one or more phenyl groups, and one or more vinyl groups. In other embodiments, the R groups individually comprise one or more fluoro groups, one or more vinyl groups, and one or more methyl groups.

In some embodiments, the silicone-based material may comprise a fluorosilicone material and the adhesive layer 104 comprises a fluorosilicone material. In other embodiments, the silicone-based material comprises silicone.

In some embodiments, the adhesive layer 104 comprises a single type of adhesive. In other embodiments, the adhesive layer 104 comprises more than one type of adhesive. In some embodiments, the adhesive layer 104 comprises a first silicone-based material comprising one type of R group and at least a second silicone-based material comprising a different type of R group. In other embodiments, the adhesive layer 104 comprises a silicone-based material comprising more than one type of R group.

In some embodiments, the adhesive layer 104 comprises a pressure sensitive adhesive (PSA). In some such embodiments, the adhesive layer 104 in configured to bond or attach to the patient responsive to application of pressure.

In some embodiments, the adhesive layer 104 is soluble in a solvent such that the adhesive layer 104 exhibits a reduction in tackiness responsive to exposure to the solvent. The solvent may comprise, for example, one or more of methyl ethyl ketone (MEK), acetone, dichloromethane (DCM) (CH₂Cl₂), isopropyl alcohol (IPA) (C₃H₈O), toluene, xylene, acetic acid (CH₃COOH), methanol, ethanol, or mineral spirits. In some embodiments, the solvent is formulated and configured to chemically react with the adhesive layer 104 over a duration of time.

The adhesive layer 104 may be formed from a two-part composition including a first part comprising a cross-linking agent and (optionally) a condensation catalyst and a second part comprising one or more polymers and (optionally) filler materials. In some embodiments, the cross-linking agent is formed of and includes an alkoxy, acetoxy ester, epoxy, or oxime silane, such as methyl trimethoxy silane, and methyl triacetoxysilane. The condensation catalyst may include one or more of organotitanate catalysts, such as tetraalkoxy titanates and chelated titanates, tin, such as dibutyl tin dilaurate (DBTDL). The polymers may comprise one or more polysiloxane or polydimethylsiloxanes. In some embodiments, the adhesive layer 104 comprises a two-part medical grade silicone adhesive.

A thickness T₂ of the adhesive layer 104 in the vertical direction (e.g., the Z-direction) may be within a range of from about 0.01 mm to about 5.0 mm, such as from about 0.01 mm to about 0.1 mm, from about 0.1 mm to about 0.5 mm, from about 0.5 mm to about 1.0 mm, from about 1.0 mm to about 2.0 mm, from about 2.0 mm to about 3.0 mm, or from about 3.0 mm to about 5.0 mm. In some embodiments, the thickness T₂ of the adhesive layer 104 is less than the thickness T₁ of the carrier film 102. In some embodiments, a thickness of the multi-layer wound dressing 101 (e.g., the combined thickness of the adhesive layer 104 and the carrier film 102 (T₂+T₁)) is about 600 μm. By way of non-limiting example, the thickness T₁ of the carrier film 102 may be within a range of from about 50 μm to about 100 μm and the thickness T₂ of the adhesive layer 104 may be within a range of from about 400 μm to about 500 μm.

The adhesive layer 104 may further include one or more of (e.g., at least one of) one or more substances to facilitate healing of the wound (e.g., healing aids), such as one or more antimicrobial materials, one or more antibiotic materials, one or more materials to reduce scarring, one or more pain relief materials, one or more anti-inflammatory materials, one or more steroids, and one or more anti-oxidants. The one or more substances may be a chemical compound or a mixture of chemical compounds.

By way of non-limiting example, the healing aids may include one or more of collagen, phytochemicals (e.g., curcumin, picroliv, arnebin-1, aloe vera, banana leaf, cocoa, tree bark, β-glucans, tumeric), and propolis. However, the disclosure is not so limited and the healing aid may include materials other than those described above.

By way of non-limiting example, the one or more antimicrobial materials may include one or more of an antiseptic agent (a biocide used to kill or inhibit growth of microorganisms present in the wound or on intact skin), silver, gold, copper, zinc oxide, titanium oxide, iodine, povidone, povidone iodine, Fe₃O₄, cephalosporins, quinolones, fluoroquinolones, aminoglycosides, beta lactams, and carbapenems. However, the disclosure is not so limited and the antimicrobial materials may include materials other than those described above.

By way of non-limiting example, the one or more antibiotic materials may include one or more of honey, zinc oxide, ciprofloxacin, ceftriaxone, tetracycline hydrochloride (TCH), amoxicillin, and silver (e.g., silver oxide, silver nitrate, silver sulfate, silver salt, silver zeolite, silver sulfadiazie (SSD), silver nanoparticles). However, the disclosure is not so limited and the anti-antibiotic materials may include materials other than those described above.

In some embodiments, the adhesive layer 104 comprises one or more antimicrobial materials and one or more antibacterial materials.

By way of non-limiting example, the one or more materials to reduce scarring may include one or more of extractum cepae (onion extract), mitomycin C, imiquimod, bleomycin, silicone, interferon, corticosteroids, and Botulinum toxin A (BTA). However, the disclosure is not so limited and the materials to reduce scarring may include materials other than those described above.

By way of non-limiting example, the one or more pain relief materials may include one or more of aspirin, capsaicin, clonidine, cannabidiol (CBD), lidocaine, and menthol. However, the disclosure is not so limited and the pain relief materials may include materials other than those described above.

In some embodiments, the one or more anti-inflammatory materials comprises one or more of hydrocortisone (HC) and carboxymethyl agarose (CMA). However, the disclosure is not so limited and the anti-inflammatory materials may include materials other than those described above.

In some embodiments, the one or more antioxidants comprise one or more of Vitamin E, sodium pyruvate, herbal antioxidants (e.g., grade seed extract), honey, and cobalt (II) complexes (CMLA).

With continued reference to FIG. 1, the multi-layer wound dressing assembly 100 includes the sacrificial liner 106 directly contacting the adhesive layer 104 of the multi-layer wound dressing 101. The sacrificial liner 106 is configured to protect the adhesive layer 104 prior to application of the multi-layer wound dressing 101 to a patient with the adhesive layer 104. In use and operation, the sacrificial liner 106 is removed prior to application of the multi-layer wound dressing 101 to a patient. After removal of the sacrificial liner 106, the adhesive layer 104 is exposed and placed into contact with the patient.

A thickness T₃ of the sacrificial liner 106 in the vertical direction (e.g., Z-direction) may be substantially the same as the thickness T₂ of the adhesive layer 104. By way of non-limiting example, the thickness T₃ of the sacrificial liner 106 may be within a range of from about 0.01 mm to about 5.0 mm, such as from about 0.01 mm to about 0.1 mm, from about 0.1 mm to about 0.5 mm, from about 0.5 mm to about 1.0 mm, from about 1.0 mm to about 2.0 mm, from about 2.0 mm to about 3.0 mm, or from about 3.0 mm to about 5.0 mm.

The transparent carrier film 102 facilitates a transparent or semitransparent film through which the wound may be viewed without removing the multi-layer would dressing 101 from the patient. In addition, the adhesive layer 104 may be substantially transparent such that the wound may be viewed through the adhesive layer 104. The multi-layer would dressing 101 facilitates drainage of the wound without removal of the multi-layer would dressing 101. The adhesive layer 104 may include the adhesive exhibiting the tackiness and may further include one or more substances (e.g., one or more of healing aids, antimicrobial materials, antibiotic materials, materials to reduce scarring, anti-inflammatory materials, steroids, anti-oxidants) to facilitate healing of the wound. The composition of the adhesive layer 104 facilitates application of the multi-layer would dressing 101 with a high tackiness and removal of the multi-layer would dressing 101 without substantially harming, further injuring or re-injuring, or causing discomfort to the patient (e.g., by pulling and/or bruising the patient). By way of comparison, conventional wound dressings are opaque and require removal to visually inspect the wound. In addition, conventional wound dressings do not exhibit a tackiness that is reduced over a duration and may cause injury during removal of the wound dressing from the patient. In some embodiments, such as for post-surgery applications, the carrier film 102 comprises a substantially impermeable material to maintain moisture between the multi-layer wound dressing 101 and the wound and promote healing of the wound. In other embodiments, the carrier film 102 comprise a permeable material (e.g., includes perforations (e.g., relatively small holes) therethrough) to facilitate air to flow through the wound and drainage of fluid to support a drying action of the wound and formation of a scab. In addition, such perforations may facilitate contacting the adhesive layer 104 with one or more materials formulated and configured to reduce the tackiness of the adhesive layer 104.

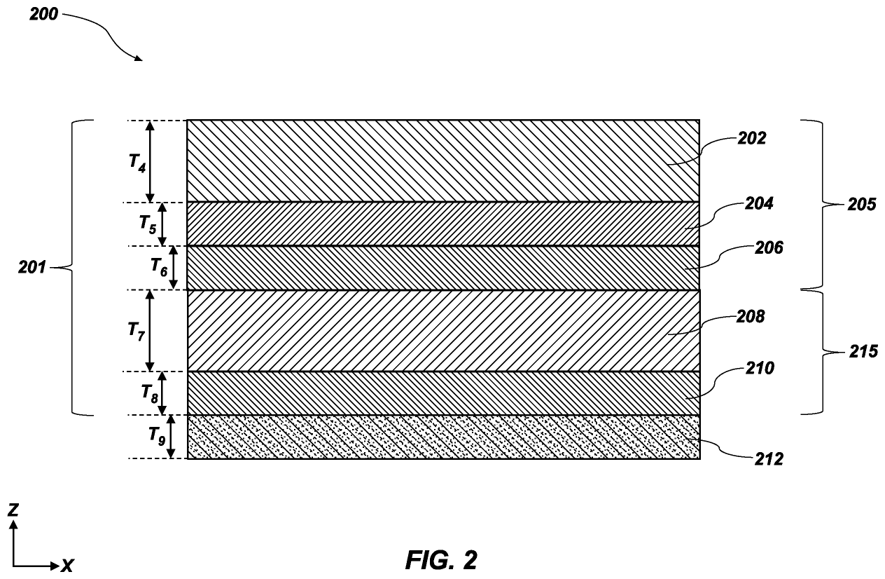

FIG. 2 is a simplified partial cross-sectional view of a multi-layer wound dressing assembly 200, in accordance with additional embodiments of the disclosure. The multi-layer wound dressing assembly 200 comprises a multi-layer wound dressing 201 and a sacrificial liner 212 vertically neighboring the multi-layer wound dressing 201. The multi-layer wound dressing 201 comprises a first portion 205 comprising an impermeable carrier film 202, an adhesive layer 204 vertically neighboring (e.g., in the Z-direction) the impermeable carrier film 202, and an absorbent material 206 vertically neighboring (e.g., in the Z-direction) the adhesive layer 204; and a second portion 215 comprising a permeable (e.g., porous) carrier film 208 vertically neighboring (e.g., in the Z-direction) the absorbent material 206, and an additional adhesive layer 210 vertically neighboring (e.g., in the Z-direction) the permeable carrier film 208. The sacrificial liner 212 vertically neighbors (e.g., in the Z-direction) the additional adhesive layer 210. The adhesive layer 204 may be vertically between (e.g., in the Z-direction) the impermeable carrier film 202 and the absorbent material 206; the absorbent material 206 may be vertically between (e.g., in the Z-direction) the adhesive layer 204 and the permeable carrier film 208; the permeable carrier film 208 may be vertically between (e.g., in the Z-direction) the absorbent material 206 and the additional adhesive layer 210; and the additional adhesive layer 210 may be vertically between (e.g., in the Z-direction) the permeable carrier film 208 and the sacrificial liner 212.

The impermeable carrier film 202 may be substantially similar to the carrier film 102 (FIG. 1). The impermeable carrier film 202 may be formed of and include one or more of the materials described above with reference to the carrier film 102. The impermeable carrier film 202 may comprise a transparent material or a semi-transparent material to facilitate visual inspection of the wound or other components of the multi-layer wound dressing 201 through the impermeable carrier film 202 without removal of the multi-layer wound dressing 201 from the patient.

In some embodiments, the impermeable carrier film 202 exhibits a tackiness less than a tackiness of other portions of the multi-layer wound dressing assembly 200 (e.g., the adhesive layer 204, the permeable carrier film 208, the additional adhesive layer) to facilitate removal of the impermeable carrier film 202 and the underlying absorbent material 206 without substantially removing other portions of the multi-layer wound dressing assembly 200 and disturbing the wound.

The impermeable carrier film 202 may have a thickness T₄ in the vertical direction (e.g., in the Z-direction) substantially similar to the thickness T₁ (FIG. 1) of the carrier film 102 (FIG. 1), such as within a range of from about 0.01 mm to about 10.0 mm.

The adhesive layer 204 may be formed of and include one or more of the materials described above with reference to the adhesive layer 104. The adhesive layer 204 may comprise a material exhibiting a desired tackiness (e.g., adhesiveness) to secure the multi-layer wound dressing 201 to the patient. The adhesive layer 204 may be formulated and configured to exhibit a relatively high tack when applied to a patient and exhibit a reduction in tackiness over a duration of time; may be configured to exhibit a reduction in tackiness responsive to exposure to one or more conditions; or both, as described above with reference to the adhesive layer 104.

A thickness T₅ of the adhesive layer 204 may be substantially similar to the thickness T₂ (FIG. 2) of the adhesive layer 104 (FIG. 1), such as within a range of from about 0.01 mm to about 5 mm.

The absorbent material 206 may be formulated and configured to facilitate removal of liquid (e.g., exudate, drainage) from the wound while the multi-layer wound dressing 201 is on the patient. The absorbent material 206 may comprise an absorbent material configured to absorb the drainage (e.g., serosanguinous drainage) from the wound. The impermeable carrier film 202 and the adhesive layer 204 hold the absorbent material 206 in place during use of the multi-layer wound dressing 201.

The absorbent material 206 may comprise a woven material, a non-woven material, or a latex material. In some embodiments, the absorbent material comprises acrylic, cellulose, cotton, foam, gauze, a polyester, or rayon. However, the disclosure is not so limited and the absorbent material 206 may comprise one or more materials other than those described above.

The absorbent material 206 may have a thickness T₆ in the vertical direction within a range of from about 0.01 mm to about 5 mm, such as from about 0.01 mm to about 0.1 mm, from about 0.1 mm to about 0.5 mm, from about 0.5 mm to about 1.0 mm, from about 1.0 mm to about 2.0 mm, from about 2.0 mm to about 3.0 mm, or from about 3.0 mm to about 5.0 mm.

The permeable carrier film 208 may comprise a permeable (e.g., porous) material configured to allow moisture to drain from the wound to the absorbent material 206. By way of non-limiting example, in some embodiments, the permeable carrier film 208 comprises perforations to facilitate flow of fluid from the wound, through the permeable carrier film 208 and to the absorbent material 206. In other words, moisture from the wound may be transferred through the permeable carrier film 208 to be absorbed by the absorbent material 206. In some embodiments, the permeable carrier film 208 comprises a different material composition than the impermeable carrier film 202. In some embodiments, the permeable carrier film 208 comprises PET. In other embodiments, the permeable carrier film 208 comprises one or more of polymethyl methacrylate (PMMA), polycarbonate (PC), polyethylene (PE), polypropylene (PP), polyvinyl chloride (PVS), and acrylonitrile-butadiene-styrene (ABS).

In some embodiments, the permeable carrier film 208 is flexible and inelastic, as described above with reference to the carrier film 102 (FIG. 1). In some embodiments, the permeable carrier film 208 is transparent or semi-transparent to facilitate visual inspection of the wound after removal of the absorbent material 206 (and the adhesive layer 204 and the impermeable carrier film 202).

A vertical (e.g., in the Z-direction) thickness T₇ of the permeable carrier film 208 may be within a range of from about 0.01 millimeter (mm) to about 10.0 mm, such as from about 0.01 mm to about 0.1 mm, from about 0.1 mm to about 0.5 mm, from about 0.5 mm to about 1.0 mm, from about 1.0 mm to about 2.0 mm, from about 2.0 mm to about 3.0 mm, from about 3.0 mm to about 5.0 mm, or from about 5.0 mm to about 10.0 mm. In some embodiments, the thickness T₇ of the permeable carrier film 208 is about the same as the thickness T₄ of the impermeable carrier film 202.

The additional adhesive layer 210 may comprise one or more of the materials described above with reference to the adhesive layer 204. In some embodiments, the additional adhesive layer 210 comprises substantially the same material composition as the adhesive layer 204. In some embodiments, the additional adhesive layer 210 comprises one or more of one or more healing aids, one or more antimicrobial materials, one or more antibiotic materials, one or more materials to reduce scarring, one or more anti-inflammatory materials, one or more steroids, and one or more anti-oxidants. In some embodiments, the adhesive layer 204 does not include one or more of the additional materials.

A vertical (e.g., in the Z-direction) thickness T₈ of the additional adhesive layer 210 may be substantially similar to the thickness T₅ of the adhesive layer 204, such as within a range of from about 0.01 mm to about 5 mm.

The sacrificial liner 212 may comprise one or more of the materials described above with reference to the sacrificial liner 106. In use and operation, the sacrificial liner 212 is removed prior to application of the multi-layer wound dressing 201 to a patient. After removal of the sacrificial liner 212, the adhesive layer 210 is exposed and placed into contact with the patient to secure the multi-layer wound dressing 201 to the patient.

A vertical (e.g., in the Z-direction) thickness T₉ of the sacrificial liner 212 may be substantially similar to the thickness T₃ (FIG. 1) of the sacrificial liner 212, such as within a range of from about 0.01 mm to about 5 mm.

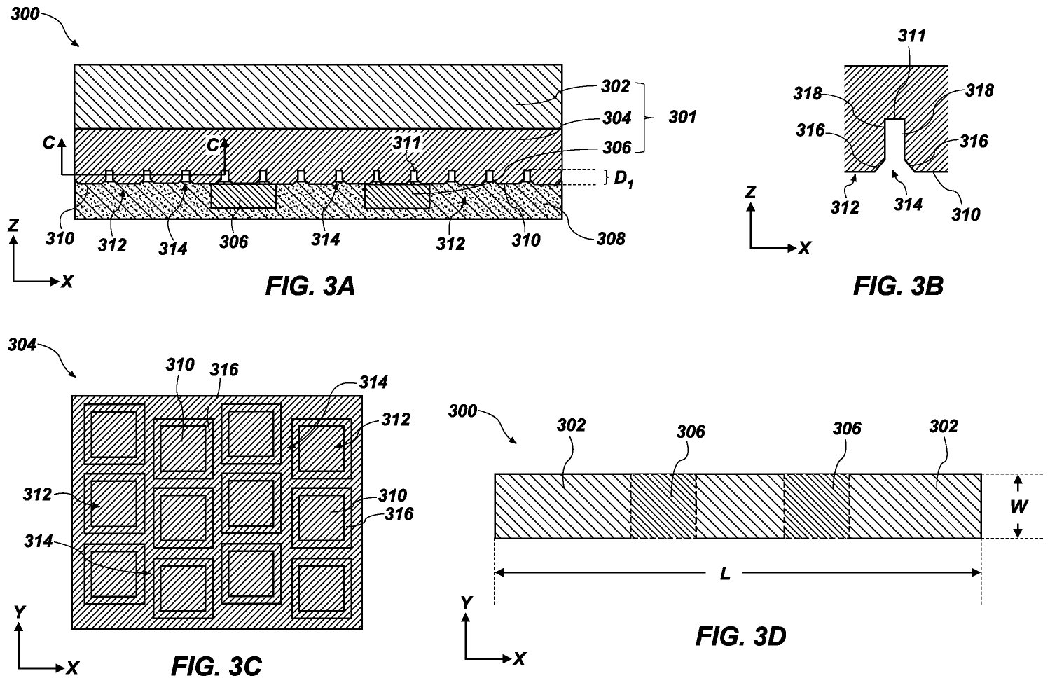

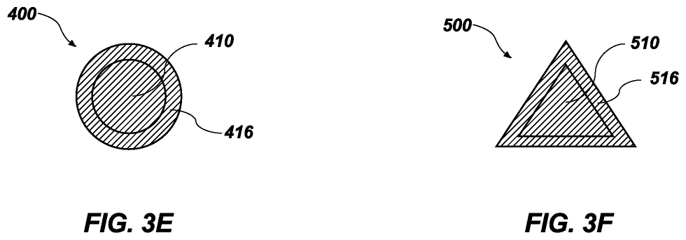

FIG. 3A is a simplified partial cross-sectional view of a multi-layer wound dressing assembly 300, in accordance with additional embodiments of the disclosure. With reference to FIG. 3A, the multi-layer wound dressing assembly 300 includes a multi-layer wound dressing 301 comprising a carrier film 302, an adhesive layer 304 vertically below (e.g., in the Z-direction) the carrier film 302, an absorbent material 306 vertically below (e.g., in the Z-direction) the adhesive layer 304, and a sacrificial liner 308 vertically below (e.g., in the Z-direction) the adhesive layer 304 and the absorbent material 306. In some embodiments, a portion of the sacrificial liner 308 is in contact with a lower surface of the adhesive layer 304 and sides of the absorbent material 306, and another portion of the sacrificial liner 308 is in contact with a lower surface of the absorbent material 306. The absorbent material 306 may include one or more portions that are horizontally spaced (e.g., in the X-direction) from one another such that portions of the adhesive layer 304 are exposed between horizontally neighboring portions of the absorbent material 306.

The carrier film 302 may be substantially the same as the carrier film 102 (FIG. 1) and may be formed of and include one or more of the materials described above with reference to the carrier film 102. The carrier film 302 may comprise a transparent material or a semi-transparent material to facilitate visual inspection of the wound or other components of the multi-layer wound dressing 301 through the carrier film 302 without removal of the multi-layer wound dressing 301 from the patient.

The carrier film 302 may have a vertical thickness (e.g., in the Z-direction) substantially the same as the thickness T₁ of the carrier film 102, such as within a range of from about 0.01 mm to about 10.0 mm.

The sacrificial liner 308 may be substantially the same as the sacrificial liner 106 (FIG. 1) and may be formed of and include one or more of the materials described above with reference to the sacrificial liner 106. In use and operation, the sacrificial liner 308 is removed prior to application of the multi-layer wound dressing 301 to a patient. After removal of the sacrificial liner 308, the adhesive layer 304 and the absorbent material 306 are exposed and placed into contact with the patient to secure the multi-layer wound dressing 301 to the patient.

The adhesive layer 304 may be formed of and include one or more of the materials described above with reference to the adhesive layer 104 (FIG. 1). The adhesive layer 304 may comprise a material exhibiting a desired tackiness (e.g., adhesiveness) to secure the multi-layer wound dressing 301 to the patient. The adhesive layer 304 may be formulated and configured to exhibit a relatively high tack when applied to a patient and exhibit a reduction in tackiness over a duration of time; may be configured to exhibit a reduction in tackiness responsive to exposure to one or more conditions; or both, as described above with reference to the adhesive layer 104.

A vertical thickness of the adhesive layer 304 may be substantially similar to the vertical thickness T₂ (FIG. 2) of the adhesive layer 104 (FIG. 1), such as within a range of from about 0.01 mm to about 5 mm.

In some embodiments, a lower portion (e.g., an exposed surface) of the adhesive layer 304 is textured. The lower portion of the adhesive layer 304 may include a lower surface 310, at least a portion of which is in contact with at least a portion of the absorbent material 306; and an upper surface 311 vertically spaced (e.g., in the Z-direction) from the absorbent material 306 and the lower surface 310.

The lower portion of the adhesive layer 304 may be at least partially defined by protrusions 312 (e.g., ridges) horizontally spaced (e.g., in the X-direction, in the Y-direction) from one another by channels 314 (e.g., trenches, valleys). The channels 314 may be located farther from the absorbent material 306 than the protrusions 312 and may be located closer to the adhesive layer 304 than the protrusions 312. The channels 314 may individually be partially defined by the upper surface 311 and sidewalls of the protrusions 312.

A vertical depth D₁ of the channels 314 may be within a range of from about 0.01 mm to about 2.0 mm, such as from about 0.01 mm to about 0.1 mm, from about 0.1 mm to about 0.5 mm, from about 0.5 mm to about 1.0 mm, or from about 1.0 mm to about 2.0 mm.

FIG. 3B is a simplified cross-sectional view illustrating an enlarged cross-section of one of the channels 314. In some embodiments, the channels 314 may exhibit a substantially square or a substantially rectangular cross-sectional shape. In some embodiments, the channel 314 may be defined by angled surfaces 316, sidewalls 318 (e.g., substantially vertical sidewalls) connected to the angled surfaces 316, and the upper surface 311 connected to the sidewalls 318. The angled surfaces 316 extend from the lower surface 310 to the sidewalls 318. The sidewalls 318 extend between and connect the angled surfaces 316 to the upper surface 311. In some embodiments, the angled surfaces 316 define the channel 314 to have a larger dimension proximate the absorbent material 306 (and the wound) than proximate the carrier film 302 (and a back side of the adhesive layer 304).

In some embodiments, the protrusions 312 exhibit a substantially rectangular cross-sectional shape. In some embodiments, the lower surface 310 and the angled surfaces 316 define an exposed surface of the protrusions 312 (e.g., the lowermost surfaces (e.g., in the Z-direction) of the protrusions 312) to exhibit a substantially rectangular cross-sectional shape, the lower surface 310 defining a relatively smaller rectangular cross-sectional shape than the angled surfaces 316.

FIG. 3C is a simplified partial cross-sectional view of the multi-layer wound dressing assembly 300 taken through section line C-C of FIG. 3A and illustrating the orientation of the protrusions 312 and the channels 314 in the lower portion of the adhesive layer 304. In some embodiments, the protrusions 312 are staggered such that protrusions 312 horizontally neighboring (e.g., in the X-direction) may be at least partially offset from one another. In other embodiments, the protrusions 312 are not staggered such that protrusions 312 horizontally neighboring (e.g., in the X-direction) one another are horizontally aligned (e.g., in the Y-direction) with one another. In some such embodiments, the protrusions 312 are individually located within horizontal boundaries (e.g., in the Y-direction) of horizontally neighboring (e.g., in the X-direction) protrusions 312.

FIG. 3D is a simplified partial top-down view of the multi-layer wound dressing assembly 300, in accordance with embodiments of the disclosure. In some embodiments, the absorbent material 306 may span substantially an entire width W (e.g., in the Y-direction) of the multi-layer wound dressing assembly 300. The width W may be within a range of from about 6.35 mm (about 0.25 inch) to about 50.8 mm (about 2.0 inches), such as from about 6.35 mm (about 6.35 mm) to about 12.7 mm (about 0.50 inch), from about 12.7 mm (about 0.5 inch) to about 25.4 mm (about 1.0 inch), from about 25.4 mm (about 1.0 inch) to about 38.1 mm (about 1.50 inch), or from about 38.1 mm (about 1.50 inches) to about 50.8 mm (about 2.0 inches). In some embodiments, the width W is within a range of from about 6.35 mm (about 0.25 inch) to about 19.1 mm (about 0.75 inch), such as about 12.7 mm (about 0.50 inch).

The absorbent material 306 may have a length L (e.g., in the X-direction) within a range of from about 50.8 mm (about 2.0 inches) to about 305.0 mm (about 12.0 inches), such as from about 50.8 mm (about 2.0 inches) to about 101.6 mm (about 4.0 inches), from about 101.6 mm (about 4.0 inches) to about 152.4 mm (about 6.0 inches), from about 152.4 mm (about 6.0 inches) to about 203.2 mm (about 8.0 inches), from about 203.2 mm (about 8.0 inches) to about 254.0 mm (about 10.0 inches), or from about 254.0 mm (about 10.0 inches) to about 305.0 mm (about 12.0 inches). In some embodiments, the length L is greater than the width W. In some embodiments, the length L is within a range of from about 50.8 mm (about 2.0 inches) to about 101.6 mm (about 4.0 inches), such as about 76.2 mm (about 3.0 inches).

The absorbent material 306 is illustrated in broken lines in FIG. 3D to illustrate that the absorbent material 306 is located vertically below (e.g., in the Z-direction) the carrier film 302 (and the adhesive layer 304 (FIG. 3B)).

With collective reference to FIG. 3A and FIG. 3D, a horizontally central (e.g., in the X-direction) of the multi-layer wound dressing assembly 300 may not include the absorbent material 306 such that a wound may be visibly viewed through the carrier film 302 and the adhesive layer 304 without removal of the multi-layer wound dressing assembly 300 (e.g., since the carrier film 302 and the adhesive layer 304 are transparent or semi-transparent and the wound is not covered (e.g., obstructed) by the absorbent material 306). In some embodiments, a horizontally central portion of the multi-layer wound dressing assembly 300 in a direction of the multi-layer wound dressing assembly 300 having a largest dimension (e.g., in the direction of the length L, in the X-direction) may be free of the absorbent material 306 such that a wound may be visibly viewed through the carrier film 302 and the adhesive layer 304 without obstruction by the absorbent material 306.

In use and operation, the channels 314 may provide a flow pathway for fluid from the wound to travel to the absorbent material 306 to be absorbed by the absorbent material 306. Since the channels 314 facilitate flow of fluid from the wound to the absorbent material 306, the channels 314 may facilitate placement of the multi-layer wound dressing 301 over a wound without covering the wound with the absorbent material 306 such that the wound is visible through the carrier film 302 and the adhesive layer 304.

In addition, the channels 314 may provide a flow pathway for a solvent or other chemical introduced to the adhesive layer 304 to contact the adhesive layer 304 and reduce the tackiness of the adhesive layer 304 (e.g., such as by chemical reaction with the adhesive layer 304).

Although the protrusions 312 and the channels 314 of FIG. 3A through FIG. 3C have been described and illustrated as exhibiting a particular shape, the disclosure is not so limited. In other embodiments, the protrusions 312 and the channels 314 exhibit a different shape. By way of non-limiting example, in some embodiments, the protrusions 312 may individually exhibit a circular cross-sectional shape, an elliptical cross-sectional shape, a triangular cross-sectional shape, or a truncated pyramidal shape.

FIG. 3E is a simplified top-down view illustrating a protrusion 400 having a circular cross-sectional shape, in accordance with embodiments of the disclosure. The protrusion 400 may replace one or more of the protrusions 312 of FIG. 3A through FIG. 3C. As described above with reference to the protrusion 312 (FIG. 3A through FIG. 3C), the protrusion 400 is defined by a lower surface 410 (some of which contact the absorbent material 306 (FIG. 3A)) and angled surfaces 416 extending from the lower surface 410 to sidewalls (e.g., sidewalls 318 (FIG. 3B)) defining the channels (e.g., channels 314 (FIG. 3A, FIG. 3B)). The lower surface 410 may have a circular cross-sectional shape and the angled surface 416 may define a relatively larger circular cross-sectional shape than defined by the lower surface 410.

FIG. 3F is a simplified top-down view illustrating a protrusion 500 having a triangular cross-sectional shape, in accordance with additional embodiments of the disclosure. The protrusion 500 may replace one or more of the protrusions 312 of FIG. 3A through FIG. 3C. As described above with reference to the protrusion 312 (FIG. 3A through FIG. 3C), the protrusion 500 is defined by a lower surface 510 (some of which contact the absorbent material 306 (FIG. 3A)) and angled surfaces 516 extending from the lower surface 410 to sidewalls (e.g., sidewalls 318 (FIG. 3B)) defining the channels (e.g., channels 314 (FIG. 3A, FIG. 3B)). The lower surface 510 may have a triangular cross-sectional shape and the angled surface 516 may define a relatively larger triangular cross-sectional shape than defined by the lower surface 510.

In some embodiments, the protrusions 500 do not include the angled surface 516 and are defined by the lower surface 510 and the sidewalls (e.g., sidewalls 318) defining the channels (e.g., channels 314 (FIG. 3A, FIG. 3B)). In some such embodiments, the lower surface 510 is connected to the upper surface (e.g., upper surface 311 (FIG. 3B)) by the sidewalls (e.g., sidewalls 318 (FIG. 3B)).

In some embodiments, the adhesive layer 304 includes protrusions 312, 400, 500 having more than one type of cross-sectional shape. In some embodiments, at least some of the protrusions 312, 400, 500 of the adhesive layer 304 exhibit a first cross-sectional shape and at least other protrusions 312, 400, 500 of the adhesive layer 304 exhibit a second, different cross-sectional shape.

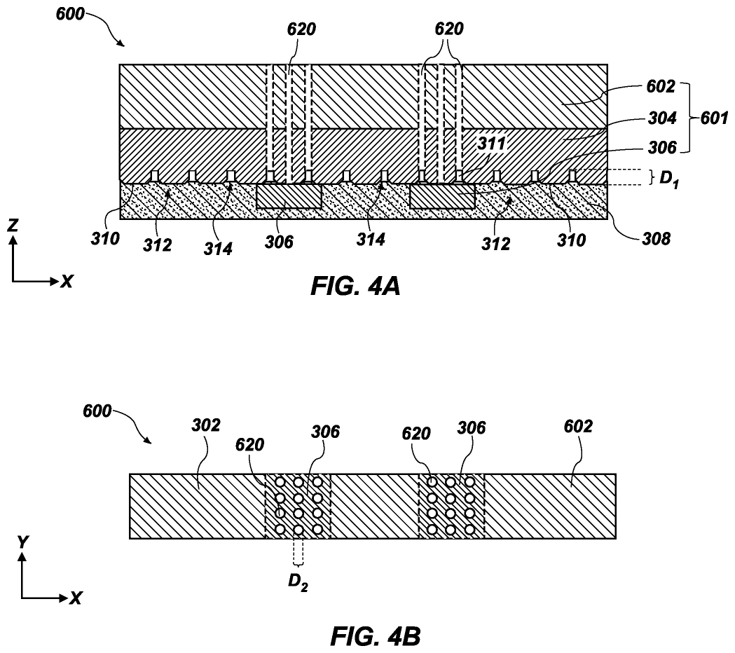

FIG. 4A and FIG. 4B are a respective partial cross-sectional view (FIG. 4A) and a partial top-down view (FIG. 4B) illustrating a multi-layer wound dressing assembly 600, in accordance with embodiments of the disclosure. The multi-layer wound dressing assembly 600 may be substantially similar to the multi-layer wound dressing assembly 300 described above with reference to FIG. 3A through FIG. 3D except that the multi-layer wound dressing assembly 600 includes a carrier film 602 including one or more perforations 620 extending vertically (e.g., in the Z-direction) therethrough, such as through the carrier film 602 and the adhesive layer 304.

The multi-layer wound dressing assembly 600 includes a multi-layer wound dressing 601 comprising the carrier film 602, an adhesive layer 304 vertically below (e.g., in the Z-direction) the carrier film 302, and an absorbent material 306 vertically below (e.g., in the Z-direction) the adhesive layer 304. The adhesive layer 304 may be substantially the same as described above with reference to FIG. 3A through FIG. 3F, except that the adhesive layer 304 includes the perforations 620 extending vertically (e.g., in the Z-direction) therethrough. The absorbent material 306 may be substantially the same as described above with reference to FIG. 3A through FIG. 3F.

The carrier film 602 may be substantially the same as the carrier film 302 described above with reference to FIG. 3A through FIG. 3F, except that the carrier film 602 includes one or more perforations 620 extending therethrough. The one or more perforations 620 may be sized, shaped, and configured to facilitate drying of the absorbent materials 306. In some such embodiments, the adhesive layer 304 comprises a permeable material such that moisture may be removed from the wound and the absorbent materials 306 through the adhesive layer 304 and the perforations 620 of the carrier film 602. In some embodiments, the perforations 620 extend through the adhesive layer 304 to facilitate removal of moisture from the wound. In some embodiments, the perforations 620 may provide a flow pathway for a solvent or other chemical introduced to the adhesive layer 304 to contact the adhesive layer 304 and reduce the tackiness of the adhesive layer 304 (e.g., such as by chemical reaction with the adhesive layer 304).

With reference to FIG. 4A and FIG. 4B, in some embodiments, the perforations 620 within the carrier film 602 are located within horizontal boundaries (e.g., in the X-direction, in the Y-direction) of the absorbent material 306. In other words, portions of the carrier film 602 and the adhesive layer 304 including the perforations 620 may be located directed vertically above (e.g., in the Z-direction) the absorbent material 306.

In some embodiments, the perforations 620 are substantially uniformly spaced from one another within regions of the carrier film 602 and the adhesive layer 304 directly vertically overlying and within horizontal boundaries of the absorbent material 306. In other embodiments, at least some of the perforations 620 are not substantially uniformly spaced.

The perforations 620 may exhibit a substantially circular cross-sectional shape. In some embodiments, each of the perforations 620 exhibit substantially the same cross-sectional shape. In other embodiments, at least one of the perforations 620 exhibits a cross-sectional shape different from the cross-sectional shape of at least one additional one of the perforations 620.

A dimension (e.g., a diameter) D₂ of the perforations 620 may be within a range of from about 0.79 mm (about 1/32 inch) to about 12.7 mm (about 0.50 inch), such as from about 0.79 mm (about 1/32 inch) to about 1.59 mm (about 1/16 inch), from about 1.59 mm (about 1/16 inch) to about 3.175 mm (about 0.125 inch), from about 3.175 mm (about 0.125 inch) to about 6.35 mm (about 0.25 inch), from about 6.35 mm (about 0.25 inch) to about 9.53 mm (about 0.375 inch), or from about 9.53 mm (about 0.375 inch) to about 12.7 mm (about 0.50 inch). In some embodiments, the diameter D2 is less than about 1.59 mm (about 1/16 inch), such as less than about 0.79 mm (about 1/32 inch).

In some embodiments, each of the perforations 620 exhibits substantially the same dimension D₂. In other embodiments, at least some of the perforations 620 exhibit a different dimension D₂ than at least one additional one of the perforations 620.

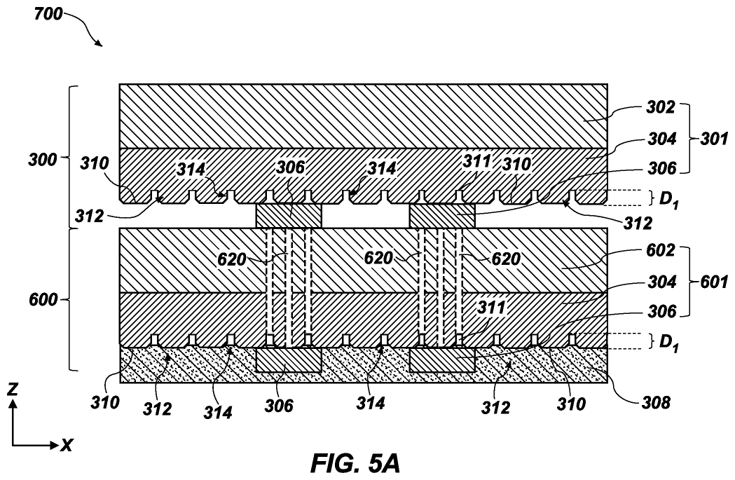

FIG. 5 is a simplified partial cross-sectional view of a multi-layer wound dressing assembly 700, in accordance with additional embodiments of the disclosure. The multi-layer wound dressing assembly 700 may include the multi-layer wound dressing assembly 600 of FIG. 4A and FIG. 4B configured to be placed on a patient and the multi-layer wound dressing assembly 300 of FIG. 3A through FIG. 3F (but not including, for example, the sacrificial liner 308) configured to be placed vertically over the multi-layer wound dressing assembly 600.

In some embodiments, the absorbent material 306 of the multi-layer wound dressings 301 is horizontally aligned (e.g., in the X-direction, in the Y-direction) with the absorbent material 306 of the multi-layer wound dressing 601. In some embodiments, the perforations 620 are located within horizontal boundaries (e.g., in the X-direction, in the Y-direction) of the absorbent material 306 of the multi-layer wound dressing 301 and the absorbent material 306 of the multi-layer wound dressing 601.

In some embodiments, the adhesive layer 304 of the multi-layer wound dressing 301 may exhibit a tackiness less than the adhesive layer 304 of the multi-layer wound dressing 601 to facilitate removal of the removal of the adhesive layer 304 of the multi-layer wound dressing 301 and removal and replacement of the absorbent material 306 between the multi-layer wound dressing 301 and the multi-layer wound dressing 601 without substantially removing the multi-layer wound dressing 601 (e.g., the absorbent material 306 of the multi-layer wound dressing 601, the adhesive layer 304 of the multi-layer wound dressing 601) from the patient and inadvertently hindering the healing process of the wound.

In some embodiments, one or more components of the multi-layer wound dressings 101, 201, 301, 601 may include one or more materials formulated and configured to exhibit a change in color to indicate the presence of an infection in the wound. By way of non-limiting example, one or more of the adhesive layers 104, 204, 304, and the additional adhesive layer 210 includes one or more materials formulated and configured to exhibit a change in color responsive to exposure to an infection. By way of non-limiting example, in some embodiments, one or more of the adhesive layers 104, 204, 304, and the additional adhesive layer 210 may be configured to exhibit a change in color responsive to a change in pH, or responsive to a chemical reaction indicating the presence of an infection. In some embodiments, wounds may exhibit a higher temperature than portions of the patient's body proximate the wound. In addition, wounds may exhibit swelling. In some embodiments, one or more of the adhesive layer 104, 204, 304 and the additional adhesive layer 210 may include a material formulated and configured to exhibit a change in color responsive to a temperature of, or a change in temperature of, the respective one of the adhesive layer 104, 204, 304 and the additional adhesive layer 210. A change of color of one or more of the adhesive layer 104, 204, 304 and the additional adhesive layer 210 may be an indication of infection of the wound. In other embodiments, one or more of the adhesive layer 104, 204, 304 and the additional adhesive layer 210 is configured to exhibit a change in color responsive to a change in pH, which may be an indication that the multi-layer wound dressing 101, 201, 301, 601 should be changed.

Accordingly, the multi-layer wound dressings 101, 201, 301, 601 may include an adhesive material (e.g., the adhesive layers 104, 204, 304 and the additional adhesive layer 210) formulated and configured to exhibit a reduced tackiness over a duration of time. In some such embodiments, the multi-layer wound dressings 101, 201, 301, 601 may be removed from a patient without harming or bruising the patient. Forming the adhesive layer 104, 204, 304 from a silicone-based material facilitates removal of the multi-layer wound dressings 101, 201, 301, 601 from a patient without re-opening a wound or harming the patient. By way of comparison, conventional adhesives are formed of an acrylic material that do not exhibit a reduction in tackiness over time (or exhibit a substantially reduced reduction in tackiness over time) and, thus, may reinjure the patient during removal of the wound dressing.

In addition, the components of the multi-layer wound dressings 101, 201, 301, 601 may be transparent or semi-transparent such that the wound or one or more other components (e.g., the absorbent material 206, 306) may be viewed through the multi-layer wound dressings 101, 201, 301, 601 without removal of the multi-layer wound dressing.

While embodiments of the disclosure may be susceptible to various modifications and alternative forms, specific embodiments have been shown by way of example in the drawings and have been described in detail herein. However, it should be understood that the disclosure is not limited to the particular forms disclosed. Rather, the disclosure encompasses all modifications, variations, combinations, and alternatives falling within the scope of the disclosure as defined by the following appended claims and their legal equivalents.

What is claimed is:

1. A multi-layer wound dressing assembly, comprising:
a carrier film comprising a transparent material or a semi-transparent material; and
an adhesive layer comprising a silicone-based material and in contact with the carrier film and exhibiting a tackiness formulated and configured to decrease over a duration of time.

2. The multi-layer wound dressing assembly of claim 1, wherein the tackiness of the adhesive layer decays over the duration of time.

3. The multi-layer wound dressing assembly of claim 1, wherein the tackiness of the adhesive layer is reduced responsive to exposure to a solvent, moisture, heat, cooling, or ultraviolet light.

4. The multi-layer wound dressing of claim 1, wherein the adhesive layer defines one or more channels.

5. The multi-layer wound dressing assembly of claim 1, wherein the adhesive layer comprises one or more of:
one or more materials configured to reduce scarring;
one or more antibiotic materials;
one or more antimicrobial materials; and
one or more pain relief materials.

6. The multi-layer wound dressing assembly of claim 1, further comprising an absorbent material on a side of the adhesive layer opposite the carrier film.

7. The multi-layer wound dressing assembly of claim 6, wherein the absorbent material contacts less than an entire surface of the side of the adhesive layer.

8. The multi-layer wound dressing assembly of claim 6, wherein a horizontally central portion of the adhesive layer is not in contact with the absorbent material.

9. The multi-layer wound dressing assembly of claim 1, wherein the carrier film is permeable.

10. The multi-layer wound dressing assembly of claim 1, wherein the carrier film is substantially impermeable.

11. The multi-layer wound dressing assembly of claim 1, wherein one of the adhesive layer and the carrier film comprise a material formulated and configured to exhibit a change in color responsive to exposure to an infection.

12. The multi-layer wound dressing assembly of claim 1, wherein the adhesive layer comprises a transparent or semi-transparent material.

13. The multi-layer wound dressing assembly of claim 1, wherein the adhesive layer comprises a textured surface.

14. A multi-layer wound dressing assembly, comprising:
a transparent carrier film;
a transparent adhesive layer on a side of the transparent carrier film; and
one or more absorbent materials on a side of the transparent adhesive layer opposite the transparent carrier film.

15. The multi-layer wound dressing assembly of claim 14, wherein a horizontally central portion of the transparent adhesive layer is free of the one or more absorbent materials.

16. The multi-layer wound dressing assembly of claim 14, wherein a portion of the transparent adhesive layer in contact with the one or more absorbent materials is textured.

17. The multi-layer wound dressing assembly of claim 14, wherein the transparent carrier film includes one or more perforations extending therethrough.

18. The multi-layer wound dressing assembly of claim 17, wherein the one or more perforations are located within horizontal boundaries of the one or more absorbent materials.

19. The multi-layer wound dressing assembly of claim 14, further comprising:
one or more additional absorbent materials on a side of the transparent carrier film opposite the transparent adhesive layer; and
an additional transparent adhesive layer on a side of the one or more additional absorbent materials opposite the transparent carrier film.

20. A multi-layer wound dressing assembly, comprising:
one or more first absorbent materials;
a first substantially transparent adhesive layer overlying the one or more first absorbent materials;
a first substantially transparent carrier film overlying the first substantially transparent adhesive layer;
one or more second absorbent materials overlying the first substantially transparent carrier firm;
a second substantially transparent adhesive layer overlying the one or more second absorbent materials; and
a second substantially transparent carrier film overlying the second substantially transparent adhesive layer.
